(19) United States
(12) Patent Application Publication
(10) Pub. No.: US 2002/0086065 A1
(43) Pub. Date: Jul. 4, 2002

(54) Methods and compositions for the benefit of those suffering from polycystic ovary syndrome with chromium complexes

(76) Inventor: David P. Katz, Dobbs Ferry, NY (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/001,684

(22) Filed: Oct. 25, 2001

Related U.S. Application Data
(60) Provisional application No. 60/244,791, filed on Oct. 31, 2000.

(51) Int. Cl. A61K 31/555; A61K 35/78; A61K 33/24

(52) U.S. Cl. 424/655; 424/769; 424/725; 514/420; 514/569; 514/184; 424/195.16

(57) ABSTRACT

Compositions comprising chromium complexes such as chromium picolinate or chromium nicotinate are administered to a subject presenting with Polycystic Ovary Syndrome. The compositions may further comprise at least one of a chelating agent, cyclooxygenase inhibitor, a mucolytic, and/or a salicin-containing herb.

### RELATED APPLICATIONS

This application claims priority to Provisional Application No. 60/244,791 entitled METHODS AND COMPOSITIONS FOR THE BENEFIT OF THOSE SUFFERING FROM POLYCYSTIC OVARY SYNDROME WITH CHROMIUM COMPLEXES filed on Oct. 31, 2000. The subject matter of the aforementioned application is hereby incorporated by reference.

### BACKGROUND OF THE INVENTION

1. Field of the Invention

The disclosed invention relates to compositions comprising chromium complexes and uses of these compositions in treating Polycystic Ovary Syndrome (PCOS).

2. Description of the Related Art

Polycystic Ovary Syndrome (PCOS) or Stein-Leventhal Syndrome affects an estimated 5% to 10% of women. The condition is characterized by 1) irregular or absent menses, 2) numerous cysts on the ovaries, 3) high blood pressure, 4) acne, 5) elevated insulin levels, insulin resistance, or type II diabetes, 6) infertility, 7) excess hair on the face or body, 8) male-pattern baldness, 9) abdominal obesity, and 10) abnormal lipid profiles.

The hallmark features of PCOS are obesity, insulin, resistance, abnormal lipid profile, excessive hair growth, anovulation, and infertility. Studies with insulin sensitizers (“glitazones”) have demonstrated some beneficial effects on this patient population with respect to these characteristics. Recently, the safety of glitazones has been challenged given the increased frequency of liver toxicity, especially for troglitazone.

Overview and Epidemiology of PCOS

About 5% to 10% of women of reproductive age have PCOS with the hallmark features of obesity, insulin resistance, abnormal lipid profile, excessive hair growth, anovulation, and infertility. The syndrome usually occurs at the onset of puberty, although it can appear in women who are in the middle of their reproductive years, and it may follow a familial inheritance pattern. Stein and Leventhal first recognized the syndrome in 1935, when they described seven patients with the characteristic signs, primarily anovulation and infertility; most of the women were hirsute, and some were obese (Am J Obstet Gynecol 29: 181-191 (1935)). Over time, Stein and Leventhal reported on approximately 100 patients.

Hirsutism and Obesity

Several studies have found that about 30% of amenorrheic and 90% of oligomenorrheic patients have PCOS. Patients with regular menses and hirsutism have a significant prevalence of polycystic-appearing ovaries, and the percentage is even higher for those women with amenorrhea or oligomenorrhea. In a study of women who considered themselves “normal,” that is, they had regular menses and no hirsutism, only eight of 123 had polycystic-appearing ovaries on ultrasound (Lancet, i:870-872 (1988)). Of women with irregular menstruation, 89% had the ultrasound criteria for this condition. Although none of the women with amenorrhea had polycystic ovaries, three had multicystic ovaries.

Insulin Impairment and Diabetes

A number of studies have demonstrated that PCOS patients have significant insulin resistance. From 20% to 40% of obese patients will develop impaired glucose tolerance (IGT) and eventually type II diabetes by the end of the fourth decade. However, not all women who develop this condition are obese. Patients with PCOS commonly have hyperinsulinemia, and therefore would also be at risk for developing IGT, whether lean or obese. Patients with PCOS make up about 10% of patients with IGT, and about 7% of patients with IGT progress to type II diabetes each year. Therefore, up to three million women in the United States are at risk for PCOS and diabetes.

A large number of patients with noninsulin-dependent diabetes develop frank insulin-dependent diabetes, and the risk for cardiovascular disease is greater in patients with glucose metabolism abnormalities. PCOS patients, who often have abnormal lipid profiles, appear to be at risk for cardiovascular disease.

Clinical Studies

A prospective, three-continent study examined how PCOS varied among Japanese, Italian, and American women (Am J Obstet Gynecol 167:1807-1812 (1992)). All had hyperandrogenism and chronic anovulation. The Japanese women had normal body weight, the Italian women were close to normal body weight, and the American women were overweight. The Japanese women typically were not hirsute, while the Italian and American women were. About 75% of all patients had polycystic ovaries on ultrasound. Each group had elevated LH and testosterone levels, and 80% demonstrated insulin resistance.

Another prospective study of women in whom the clinical diagnosis of PCOS was based on hyperandrogenism and chronic anovulation revealed elevated LH in only 70% to 75% (Fertil Steril 39:674-678 (1983)). A study by Bridges and colleagues showed that the emergence of polycystic ovaries starts at age six or seven and peaks at puberty, with a prevalence of around 25% (Fertil Steril 60:456-460 (1993)). This does not mean that 25% of girls have PCOS; polycystic ovaries in a woman who is destined to have this problem emerge very early, but this should not be confused with PCOS, a syndrome in which this symptom may be only one component, albeit an important one.

About 30% of patients with PCOS have normal menses. But it is unusual for a woman who has the clinical syndrome, and perhaps polycystic ovaries, to have consistently normal ovulatory menses. Many women who report normal menses have anovulatory cycles. The majority of patients with the “full-blown clinical disorder” have polycystic ovaries on ultrasound to some degree. This syndrome also encompasses a spectrum of various degrees of endocrine manifestations. Whereas only 10% or fewer patients with PCOS have normal ovaries, virtually 100% of patients with severe adrenal androgen excess manifest polycystic ovaries.

Insulin Resistance and PCOS

The association between insulin resistance and hyperandrogenism was first described in 1921 by Achard and Thiers (Bull Acad Nat'l Med 86:51-64 (1921)). Diagnosticians again became interested in this association when Kahn and Flier reported on adolescent girls with extreme insulin resistance, diabetes mellitus, and true virilization (“type A syndrome”) (N Engl J Med 294:739-745 (1976)). In 1974, Givens noted that patients with acanthosis nigricans had high insulin levels (J Clin Endocrinol Metab 38:347-355 (1974)), and Dunaif has found that many PCOS patients, both obese and lean, have acanthosis nigricans (Endocr. Rev. 18:744-800). These raised, velvety skin lesions, which may or may not be hyperpigmented, is a marker for insulin resistance.

In addition, to glucose metabolism, insulin regulates protein and lipid synthesis, as well as gene transcription. In PCOS, an abnormality occurs in how the insulin receptor transmits signals, a very early abnormality compared with type II diabetes. No mutations are present; some researchers believe that a serine kinase turns off the insulin receptors.

In 1980, Burghen and colleagues were the first to show that women with PCOS were hyperinsulinemic and that this was not necessarily associated with body weight (J Clin Endocrinol Metab 50:113-116 (1980)). In a prospective study, Dunaif subgrouped hyperandrogenic women by ovulatory status (J Clin Endocrinol Metab 65:499-507 (1987)), basing the diagnosis of PCOS on hyperandrogenism and anovulation. Whether obese or lean, women with PCOS had elevated insulin responses to oral glucose. The ovulatory hyperandrogenic women did not have hyperinsulinemia.

Women with PCOS are at increased risk for developing IGT and type II diabetes mellitus, both risk factors for cardiovascular disease. The risk rises linearly with two-hour glucose levels, and about 10% of IGT in premenopausal women is related to PCOD, and internists often find histories of PCOS in diabetic patients.

Androgens and Insulin Resistance

Does hyperandrogenism cause hyperinsulinemia, or does hyperinsulinemia cause hyperandrogenism? Researchers looked for an answer by suppressing insulin levels. Nestler did this first using diazoxide, and subsequent researchers have used metformin, weight loss, and troglitazone, an insulin sensitizer that reduces circulating insulin levels (J Clin Endocrinol Metab 68:1027 (1989)). A study by Dunaif using troglitazone showed increased insulin sensitivity that resulted in regular menses as early as one month later.

Despite conflicting results, studies have noted a connection between androgens and insulin resistance. For example, lowering androgen levels in PCOS patients who are not severely insulin resistant or obese improves insulin sensitivity. If virilizing doses of testosterone are given to women (as with female-to-male transsexuals), decreased insulin sensitivity results, although not of the magnitude seen in women with PCOS.

Hyperandrogenism causes some insulin resistance, and insulin resistance causes moderate hyperandrogenism, although this does not completely explain the association between insulin resistance and PCOS. Studies have shown that when insulin levels decrease, adrenal androgen, dehydroepiandrosterone sulfate (DHEAS), estrogen, and LH levels also decrease, suggesting that insulin is a “general augmentor” of steroidogenesis and LH secretions.

The most compelling evidence indicates genetic susceptibility. Polycystic ovary morphology is transmitted within families, and hyperandrogenism with regular ovulation, as well as PCOS and insulin resistance, also are familial. About 50% of sisters of PCOS women have some form of the syndrome, either hyperandrogenism alone or associated with chronic anovulation.

One hypothesis is that an abnormal serine kinase in PCOS phosphorylates the insulin receptor and the rate-limiting enzyme for androgen synthesis, P450c17. Insulin resistance then augments hyperandrogenism, and androgens augment insulin resistance, producing the syndrome. A second hypothesis is that some women are genetically predisposed to polycystic ovaries and some to insulin resistance. When both genes are present, they develop PCOS (Endocr Rev 18:774-800 (1997)).

Drug Therapies

“Glitazones” decrease insulin resistance by targeting nuclear peroxisome proliferator activated receptors (PPARs) to reduce fatty acid and glucose output and reduce triglyceride synthesis in the liver while increasing glucose uptake in skeletal muscle—a fundamental problem for women with PCOS. It has no effect on pancreatic beta cell insulin secretion and does not cause lactic acidosis.

Metformin decreases production and uptake of glucose without causing hypoglycemia, but it does rarely cause lactic acidosis, particularly in those with impaired renal function; it also frequently causes abdominal discomfort. Studies have attempted to determine which effects of metformin are caused by weight loss and which are caused by its impact on glucose disposal and insulin dynamics.

In one study, Ehrmann and colleagues treated PCOS in 14 women with metformin, 850 mg three times a day for 12 weeks (J Clin Endocrinol and Metabol 82:524-530 (1997). Body mass index (BMI) was unchanged, and insulin secretion was not improved. Free testosterone, stimulated LH, follicle-stimulating hormone (FSH), 17-hydroxy progesterone, androstenedione, DHEAS, progesterone, and estradiol levels were unchanged, suggesting that this is not a promising drug for use in PCOS women with insulin resistance.

Ehrmann also treated 13 obese, hyperandrogenic women with PCOS and IGT with 400 mg troglitazone a day for 12 weeks (J Clin Endocrinol and Metabol 82:2108-2116 (1997)). Fasting and two-hour glucose levels declined significantly, with reduced hemoglobin A-1C, improved insulin sensitivity, decreased androgenic hormones, and stimulated 17-hydroxyprogesterone, without any change in gonadotropins.

In a three-month, double-blind, randomized study by Dunaif et al., 25 obese, hyperandrogenic women with PCOS were given troglitazone 200 mg or 400 mg a day (J Clin Endocrinol and Metab 81:3299-3306 (1996)). Within a month, BMI was unchanged; insulin sensitivity increased; and free testosterone, estradiol, and estrone decreased. The improvement in insulin sensitivity resulted in regular menses as early as one month later. In those given 400 mg per day, androstenedione and LH decreased and sex hormone-binding globulin (SHBG) increased; FSH levels were unchanged, but two women ovulated.

Chromium is a nutritionally essential trace element. The essentiality of chromium in the diet was established in 1959 by Schwartz, as cited in Present Knowledge in Nutrition, page 571, fifth edition (1984, the Nutrition Foundation, Washington, D.C.). Chromium depletion is characterized by the disturbance of glucose, lipid and protein metabolism and by a shortened lifespan. Chromium is essential for optimal insulin activity in all known insulin-dependent systems (Boyle et al., Southern Med. J 70:1449-1453, 1977). Insufficient dietary chromium has been linked to both maturity-onset diabetes and to cardiovascular disease.

The principal energy sources for the body are glucose and fatty acids. Chromium depletion results in biologically ineffective insulin and compromised glucose metabolism. Under these conditions, the body must rely primarily on lipid metabolism to meet its energy requirements, resulting in the production of excessive amounts of acetyl-CoA and ketone bodies. Some of the documented acetyl-CoA is converted to increased cholesterol biosynthesis, resulting in hypercholesterolemia. Diabetes mellitus is characterized in large part by glycosuria, hypercholesterolemia, and often ketoacidosis. The accelerated atherosclerotic process seen in diabetics is associated with hypercholesterolemia (Boyle et al., supra.).

Dietary supplementation of chromium to normal individuals has been reported to lead to improvements in glucose tolerance, serum lipid concentrations, including high-density lipoprotein cholesterol, insulin and insulin binding (Anderson, Clin. Psychol. Biochem. 4:31-41, 1986). Supplemental chromium in the trivalent form, e.g. chromic chloride, is associated with improvements of risk factors associated with adult-onset (Type II) diabetes and cardiovascular disease.

Chromium functions as a cofactor for insulin. It binds to the insulin receptor and potentiates many, and perhaps all, of its functions (Boyle et al., supra.). These functions include, but are not limited to, the regulation of carbohydrate and lipid metabolism. (Present Knowledge in Nutrition, supra, at p. 573-577). The introduction of inorganic chromium compounds per se into individuals is not particularly beneficial. Chromium must be converted endogenously into an organic complex or must be consumed as a biologically active molecule. Only about 0.5% of ingested inorganic chromium is assimilated into the body (Recommended Daily Allowances, Ninth Revised Edition, The National Academy of Sciences, page 160, 1980). Only 1-2% of most organic chromium compounds is assimilated into the body.

U.S. Pat. No. Re. 33,988 discloses that when selected essential metals, including chromium, are administered to mammals as exogenously synthesized coordination complexes of picolinic acid, they are directly available for absorption without competition from other metals. This patent describes a composition and method for selectively supplementing the essential metals in the human diet and for facilitating absorption of these metals by intestinal cells. These complexes are safe, inexpensive, biocompatible and easy to produce. These exogenously synthesized essential metal coordination complexes of picolinic acid (pyridine-2-carboxylic acid) have the following structural formula:

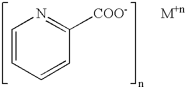

wherein M represents the metallic cation and n is equal to the cation's valence. For example, when M is Cr and n=3, then the compound is chromic tripicolinate. Other chromium picolinates disclosed include chromic monopicolinate and chromic dipicolinate.

The U.S. Recommended Daily Intake (RDI) of chromium is 120 μg. U.S. Pat. No. 5,087,623, the entire contents of which are hereby incorporated by reference, describes the administration of chromic tripicolinate for the treatment of adult-onset diabetes in doses ranging from 50 to 500 μg. International Patent Application No. WO96/35421 discloses the use of high doses of chromic tripicolinate (providing 1,000-10,000 μg chromium/day) for reducing hyperglycemia and stabilizing the level of serum glucose in humans with Type II diabetes. U.S. Pat. No. 5,789,401 discloses a chromic tripicolinate-biotin composition and its use in lowering blood glucose levels in humans with Type II diabetes.

U.S. Pat. Nos. 5,087,623; 5,087,624; and 5,175,156, the entire contents of which are hereby incorporated by reference, disclose the use of chromium tripicolinate for supplementing dietary chromium, reducing hyperglycemia and stabilizing serum glucose, increasing lean body mass and reducing body fat, and controlling blood serum lipid levels, including the lowering of undesirably high blood serum LDL-cholesterol levels and the raising of blood serum HDL-cholesterol levels. U.S. Pat. Nos. 4,954,492 and 5,194,615, the entire contents of which are hereby incorporated by reference, describe a related complex, chromic nicotinate, which is also used for supplementing dietary chromium and lowering serum lipid levels. Picolinic acid and nicotinic acid are position isomers having the following structures:

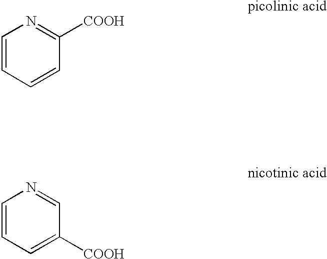

Nicotinic acid and picolinic acid form coordination complexes with monovalent, divalent and trivalent metal ions and facilitate the absorption of these metals by transporting them across intestinal cells and into the bloodstream. Chromium absorption in rats following oral administration of CrCl₃ was facilitated by the non-steroidal anti-inflammatory drugs (NSAIDs) aspirin and indomethacin (Davis et al., J. Nutrition Res. 15:202-210, 1995; Kamath et al., J. Nutrition 127:478-482, 1997). These drugs inhibit the enzyme cyclooxygenase which converts arachidonic acid to various prostaglandins, resulting in inhibition of intestinal mucus formation and lowering of intestinal pH which facilitates chromium absorption.

The present invention provides a novel method for treating Polycystic Ovary Syndrome.

### SUMMARY OF THE INVENTION

The disclosed invention is directed to a method of treating Polycystic Ovary Syndrome (PCOS). The method includes identifying a subject suffering from PCOS and administering to the subject a pharmaceutically effective dose of a composition which includes a chromium complex. Preferably, the chromium complex is chromium picolinate and/or chromium polynicotinate. Advantageously, the composition includes a chelating agent. The chelating agent can be picolinic acid, nicotinic acid, or both picolinic acid and nicotinic acid.

In another aspect, the method further includes the administration of a cyclooxygenase inhibitor. The cyclooxygenase inhibitor may include indomethacin, ibuprofen, acetaminophen and naproxen. Preferably, the composition may include a mucolytic. In a preferred embodiment, the mucolytic is guaifenesin.

In still another aspect of the invention, a subject may be administered a composition including a salicin-containing herb such as Boswellia serrata (frankincense), Betula lenta (sweet birch), Betula pubescens (white birch), Filipendula ulmaria (meadowsweet), Gautheria procumbens (wintergreens), Polulus balsamifera, Populus jackii (bahn of Gilead) or Salix alba (white willow).

Advantageously, the effective dose of the composition is between about 50 and about 10,000 micrograms.

In yet another aspect of the invention, the composition is incorporated into a pharmaceutically acceptable carrier selected from the group consisting of a tablet, capsule, microbead, emulsion, powder, granule, suspension, syrup, and elixir. The microbead can be a sugar beadlet or microcrystalline cellulose beadlet. Preferably, the chromium complex is coated on the beadlet.

### DETAILED DESCRIPTION OF THE PREFERRED EMBODIMENT

The disclosed invention relates to compositions for use in the treatment of Polycystic Ovary Syndrome (PCOS). Additionally, methods for treating PCOS are likewise contemplated. The primary basis of the present invention is the novel and unexpected discovery that chromium complexes lower blood glucose levels, thereby ameliorating some of the symptoms associated with PCOS. Chromium picolinates, for example, are insulin sensitizers with no known toxicity. The use of these compounds would be a great boon to the treatment of PCOS.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the invention herein described.

The present invention provides compositions comprising chromium complexes for the treatment of PCOS. As used herein, the term “chromium complexes” or “chromium complex” includes, without limitation, chromium picolinate, chromic tripicolinate, chromium nicotinate, chromic polynicotinate, chromium chloride, chromium histidinate, and chromium yeasts. In a preferred embodiment, the chromium complexes are synthetic. The chromium complexes facilitate absorption of chromium by intestinal cells.

While chromium complexes aid in the absorption of chromium by intestinal cells, in some embodiments, uncomplexed chelating agents are advantageously included in the compositions to facilitate absorption of other metals including, but not limited to, copper, iron, magnesium, manganese, and zinc. Suitable chelating agents include picolinic acid, nicotinic acid, or both picolinic acid and nicotinic acid. Thus, the compositions of the disclosed invention are readily absorbable forms of chromium which also facilitate absorption of other essential metals in the human diet.

The chromium complexes of the disclosed invention have the same uses as described for chromic tripicolinate in U.S. Pat. Nos. 5,087,623, 5,087,624 and 5,174,156, namely supplementing dietary chromium, lowering blood glucose levels in diabetics, lowering serum lipid levels and increasing lean body mass. Additionally, the chromium complexes of the present invention act to treat symptoms associated with PCOS.

The synthesis and use of chromium picolinates is described in U.S. Pat. Nos. Re33,988 and 5,087,623. Chromic tripicolinate is available from health food stores, drug stores and other commercial sources. The synthesis and use of chromic polynicotinate is described in U.S. Pat. No. 5,194,615.

The uncomplexed chelating agents such as picolinic acid and nicotinic acid are available from many commercial sources, including Sigma-Aldrich (St. Louis, Mo.) (picolinic acid; catalog No. P5503; nicotinic acid; catalog No. PN4126). Preferably, the ratio of the chromium complex to the chelating agent from about 10:1 to about 1:10 (w/w), more preferably from about 5:1 to about 1:5 (w/w).

The compositions of the disclosed invention are prepared by incorporating the components into a pharmaceutically acceptable carrier, including but not limited to tablets, capsules and microbeads, preferably sugar beadlets or microcrystalline cellulose.

For oral administration, the chromium complex may be incorporated into a tablet, aqueous or oil suspension, dispersible powder or granule, microbead, emulsion, hard or soft capsule, syrup or elixir. The components of the composition may also be administered separately. Compositions may be prepared according to any method known in the art for the manufacture of pharmaceutically acceptable compositions and such compositions may contain one or more of the following agents: sweeteners, flavoring agents, coloring agents and preservatives. Tablets containing the active ingredients in admixture with non-toxic pharmaceutically acceptable excipients suitable for tablet manufacture are acceptable. “Pharmaceutically acceptable” means that the agent should be acceptable in the sense of being compatible with the other ingredients of the formulation (as well as non-injurious to the individual). Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets may be uncoated or may be coated with known techniques to delay disintegration and absorption in the gastrointestinal tract and thereby provide a sustained action over a longer period of time. For example, a time delay material such as glyceryl monostearate or glyceryl stearate alone or with a wax may be employed.

In another preferred embodiment, tablets, capsules or microbeads are coated with an enteric coating which prevents dissolution in the acidic environment of the stomach. Instead, this coating dissolves in the small intestine at a more neutral pH. Because certain chromium complexes may be more stable at this neutral pH than at the acidic pH of the stomach, enhanced absorption occurs because the chromium complexes remain substantially intact until they reach the small intestine. Such enteric coated compositions are described by Bauer et al., Coated Pharmaceutical Dosage Forms: Fundamentals, Manufacturing Techniques, Biopharmaceutical Aspects, Test Methods and Raw Materials, CRC Press, Washington, D.C., 1998, the entire contents of which are hereby incorporated by reference.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions may contain the chromium complexes of the invention in admixture with excipients for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agent, such as those set forth above, and flavoring agents may be added to provide a palatable oral preparation. These compositions may be preserved by an added antioxidant such as ascorbic acid. Dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, may also be present.

Syrups and elixirs may be formulated with sweetening agents, such as glycerol, sorbitol or sucrose. Such formulations may also contain a demulcent, a preservative, a flavoring or a coloring agent.

The oral formulations described above may also include aspirin (acetylsalicylic acid), other salicylates, or another NSAID such as indomethacin, ibuprofen, acetaminophen, naproxen or any drug capable of inhibiting the cyclooxygenase pathway leading to prostaglandin synthesis. This results in a decrease in intestinal mucus production and lower intestinal pH which facilitates absorption of the chromium compositions of the present invention. The oral compositions may further include mucolytics such as guaifenesin and the like, to inhibit intestinal mucus production, and/or acids such as ascorbic acid, citric acid and the like to lower intestinal pH. Inclusion of one or both of these compounds further enhances chromium absorption. There are two forms of cyclooxygenase (cox), cox1 and cox2, which differ in their sensitivity to inhibition by NSAIDs. The cox2 isozyme promotes prostaglandin formation at sites of inflammation, but not at other sites such as the gastrointestinal tract. In contrast, relatively selective inhibition of cox1 facilitates chromic tripicolinate and chromic polynicotinate absorption. Although the selective inhibition of cox1 is desirable, any inhibitor or cox1 or cox2 can be formulated with the chromic tripicolinate and chromic polynicotinate compositions of the invention. Cox inhibitors, acids and mucolytics may also be coadministered with the chromic tripicolinate and chromic polynicotinate compositions of the invention. The amount of these drugs formulated with or coadministered with the chromic tripicolinate compositions of the invention are as follows: cox inhibitions, between about 50 mg and 500 mg; mucolytics, between about 10 mg and 250 mg; and acids, between about 50 mg and about 1,000 mg.

The coadministration or formulation of salicylate-containing herbs with the compositions of the invention is also contemplated. Class I herbs, as documented in the American Herbal Products Association's Botanical Safety Handbook (herbs that can be safely consumed when used appropriately), such as Boswelia serrata (frankincense), Betula lenta (sweet birch), Betula pubescens (white birch), Filipendula ulmaria (meadowsweet), Gaultheria procumbens (wintergreens), Populus balsamifera and Populus jackii (balm of Gilead), and Salix alba (white willow) are all salicin-containing plants with salicylate-like properties. These herbs suppress prostaglandin synthesis by cox inhibition, thereby improving absorption of the chromium complexes of the invention. These herbs are relatively free from gastric ulcerogenic effects (Singh et al., Agents and Actions 18:407-412, 1986). In addition, pre-clinical acute toxicity studies have shown that salicin-containing plants do not cause hematological disturbances (American Herbal Products Association, Botanical Safety Handbook, 1997).

The compounds and herbs described above all effect gut physiology by inhibiting prostaglandin synthesis, decreasing mucus production, and lowering gastrointestinal pH. The inclusion of these compounds, as well as an enteric coating, into the oral chromium complex compositions of the invention results in a multicomponent delivery system which allows delivery of these agents to the gastrointestinal tract where they work in concert to facilitate chromium absorption.

In a preferred embodiment, the chromium complex is coated onto microbeads. In a particularly preferred embodiment, these microbeads are sugar beadlets of various sizes, also known as nonpareils, and are commercially available from, for example, SmithKline Beecham. If the microbeads are to be used to administer the compositions of the invention to diabetic patients, the administration of other types of microbeads, such as microcrystalline cellulose, is preferred. Microcrystalline cellulose is commercially available and can be processed into beadlets of various sizes by micronization, a technique well known in the art. The microbeads are essentially a carrier for the compositions of the invention. For a description of coated beadlets, see, for example, Carstensen, J. T., Pharmaceutical Principles of solid Dosage Forms, Technonic Publishing Co., Inc., Lancaster, Pa., pp. 228-230, 1993, hereby incorporated by reference. Aqueous solutions containing the chromium complexes with or without the chelating agent components such as nicotinic acid and picolinic acid are sprayed onto the microbeads by well known methods, by suspending the microbeads in an upcurrent of air and introducing a fine spray of the active ingredients which form a coating on the outside of the microbeads which is then allowed to dry. The desired chromium complex components with or without a chelating agent may be combined into one same solution or applied using separate solutions. Optionally, the coated microbeads can be further coated with a substance to protect the active ingredients coated onto the beads, such as latex. The microbeads may be placed in a capsule prior to administration. In another preferred embodiment, the capsule or the microbeads are coated with an enteric coating to delay dissolution until reaching the small intestine.

Typically, the dosage range of chromium administered to an individual in the form of a chromium complex provides between about 50 and 10,000 micrograms per day of chromium; preferably between about 100 and 1,000 micrograms per day; more preferably, between about 200 and 500 micrograms per day. It is advantageously a pharmaceutically effective dose; i.e., it treats or reduces at least one symptom of PCOS.

In some embodiments, methods of treating PCOS with chromium complexes is contemplated. Optionally, the methods of treatment additionally include the administration of at least one uncomplexed chelating agent. The compounds of the present invention can be administered separately or as a single composition. Advantageously, a subject is administered a pharmaceutically effective dose of a chromium complex. In one embodiment, the uncomplexed chelating agent is administered substantially simultaneously. In an alternative embodiment, the chromium complex is administered first and then the uncomplexed chelating agent is administered. In yet another embodiment, the uncomplexed chelating agent is administered first. If administered separately, the compounds should be given in a temporally proximate manner, e.g. within a twenty-four hour period, such that the reduction of symptoms associated with PCOS is enhanced. More particularly, the compounds may be given within one hour of each other. The administration can be by any of the methods of administration described above or by drug delivery methods known by one of skill in the art.

The following example teaches the methods and compositions disclosed herein for ameliorating symptoms associated with PCOS through the administration of at least one chromium complex. As illustrated in the following example, the composition may optionally include picolinic acid, nicotinic acid, or both picolinic acid and nicotinic acid in combination with a chromium complex. This example is illustrative only and is not intended to limit the scope of the invention disclosed herein. The treatment method described below can be optimized using empirical techniques well known to those of ordinary skill in the art. Moreover, artisans of skill would be able to use the teachings described in the following examples to practice the full scope of the invention disclosed herein.

### EXAMPLE

A subject presenting with PCOS is identified. The subject is orally administered a tablet comprising chromium picolinate and picolinic acid at a ratio of 1:10 w/w at a daily dose of 1,000 μg of chromium. The tablet additionally comprises guaifenesin and ibuprofen in a pharmaceutically effective dose. Over the course of several weeks, a decrease in body mass and improved lipid profile is observed. The chromic picolinate sensitizes the subject's insulin and the symptoms of PCOS are reduced.

It will be appreciated that although specific embodiments of the invention have been described herein for purposes of illustration, various modifications may be made without deviating from the spirit and scope of the invention. Accordingly, the invention is not limited except as by the appended claims.

What is claimed is:

1. A method of treating Polycystic Ovary Syndrome (PCOS) comprising:
identifying a subject suffering from PCOS; and
administering to said subject an effective dose of a composition comprising at least one chromium complex.

2. The method of claim 1, wherein said chromium complex is selected from the group consisting of chromium picolinate, chromic tripicolinate, chromium nicotinate, chromic polynicotinate, chromium chloride, chromium histidinate, and chromium yeasts.

3. The method of claim 1, wherein said composition further comprises at least one chelating agent.

4. The method of claim 3, wherein said chelating agent is picolinic acid, nicotinic acid, or both.

5. The method of claim 4, wherein said chromium complex and said chelating agent are administered in a ratio of between about 1:10 and about 10:1 (w/w).

6. The method of claim 1, further comprising administering a cyclooxygenase inhibitor.

7. The method of claim 6, wherein said cycloxygenase inhibitor is selected from the group consisting of indomethacin, ibuprofen, acetaminophen, and naproxen.

8. The method of claim 1, further comprising administering a mucolytic.

9. The method of claim 8, wherein said mucolytic is guaifenesin.

10. The method of claim 1, further comprising administering a salicin-containing herb.

11. The method of claim 10, wherein said salicin-containing herb is selected from the group consisting of Boswellia serrata (frankincense), Betula lenta (sweet birch), Betula pubescens (white birch), Filipendula ulmaria (meadowsweet), Gautheria procumbens (wintergreens), Polulus balsamifera, Populus jackii (balm of Gilead) and Salix alba (white willow).

12. The method of claim 1, wherein said effective dose is between about 50 and about 10,000 micrograms.

13. The method of claim 1, wherein said composition is incorporated into a pharmaceutically acceptable carrier selected from the group consisting of a tablet, capsule, microbead, emulsion, powder, granule, suspension, syrup, and elixir.

14. The method of claim 1, wherein said composition is incorporated into a microbead.

15. The method of claim 14, wherein said microbead is a sugar beadlet or microcrystalline cellulose beadlet and said chromium complex is coated on said beadlet.
